(19) United States
(12) Patent Application Publication
(10) Pub. No.: US 2019/0197920 A1
(43) Pub. Date: Jun. 27, 2019

(54) APPARATUS AND METHOD FOR OPTICAL ULTRASOUND SIMULATION

(71) Applicants: Gustavo ABELLA, Guayaquil (EC); Ricardo Enrique GAYOSO, Buenos Aires (AR)

(72) Inventors: Gustavo ABELLA, Guayaquil (EC); Ricardo Enrique GAYOSO, Buenos Aires (AR)

(21) Appl. No.: 16/329,632

(22) PCT Filed: Aug. 30, 2017
(86) PCT No.: PCT/US2017/049427

§ 371 Date: Feb. 28, 2019

Related U.S. Application Data
(60) Provisional application No. 62/381,225, filed on Aug. 30, 2016.

(51) Int. Cl. G09B 23/28; A61B 8/00; A61B 8/08; G16H 40/63

(52) U.S. Cl. CPC G09B 23/286; G16H 40/63; A61B 8/523; A61B 8/58

(57) ABSTRACT

A training method for imaging includes obtaining a dataset volume for an organ of a patient in an ultrasound imaging mode, and providing a template associated with the dataset volume to an optical ultrasound system location. The method also includes providing a virtual transducer with a label thereon to the optical ultrasound system location. A webcam is attached to the template. The webcam is positioned to view the label on the virtual transducer. The method includes determining the planar position of the virtual transducer from optical information obtained from the webcam image of the label. Once the planar position is determined, the planar position of the virtual transducer is related to the corresponding plane in the dataset. The plane related to the dataset is then displayed at the displaying the plane from the dataset at the optical ultrasound system location.

### RELATED APPLICATION

This application claims the benefit under 35 U.S.C. § 119(e) of prior U.S. Provisional Patent Application No. 62/381,225, filed on Aug. 30, 2017, which is incorporated herein by reference.

### BACKGROUND OF THE INVENTION

Congenital anomalies have become the leading cause of infant mortality in Caucasians in the United States. Prenatal diagnosis of congenital anomalies has become an integral part of prenatal care. Many pregnant women and their families expect that their unborn child will be evaluated to ensure that it is normal. An ultrasound system is one of the most common type of evaluation systems. These systems are not cheap. In addition, these systems are not readily available in all nations. Many rural areas are not equipped with such systems. To get an ultrasound evaluation, the patient may have to travel to a center with the appropriate equipment. In some instances, there may not even be a qualified operator that can capture the appropriate images needed to perform an evaluation. To conduct an appropriate evaluation, an operator needs to extract and generate standard diagnostic planes that will provide clinically relevant information. For example, to evaluate a fetal heart, the operator needs to extract and generate standard cardiac diagnostic planes that will provide clinically relevant information. However, there are a large number of planes contained within the volume dataset, and an operator can easily get “lost” trying to obtain the standard planes to determine whether a fetal heart is normal or not.

Training operators, such as sonographers, generally takes a long period of time. Currently training is conducted at the center where ultrasonic system is located. The training really is on the job training and is obtained after working with a large number of patients. In rural areas, an ultrasonic system may be available at some central, distant site, but there may be no regular operators. As a result, someone must be trained to effectively operate the ultrasonic system so that an effective diagnosis can be made. In these situations, the resource is scarce and so there is little time to learn on the job. The operator or sonographer might not have the time to learn on the job. The resource must be shared by many so the resource may very well be much less available. There may be no time to learn on the job since the resource will be needed by many others.

An additional problem with providing such training is that materials needed for training may be scarce. If the training materials have to be delivered, that too can be a problem. Delivery to many rural or remote areas may also be spotty and take a long time. In some instances, mailed items may never arrive. In summary, there are a myriad of problems associated with getting training materials to certain persons in need of training. Furthermore, operator training must be tailored for students of any level since operators may be non-medical professionals, in addition to a nurse, doctor, or professional.

### SUMMARY OF THE INVENTION

Described is an apparatus and method for training people to operate fetal intelligent navigation echocardiography (FINE). Use of this apparatus and method allows for visualization of standard fetal echocardiography views from dataset volumes obtained with spatiotemporal image correlation (STIC).

A training method for imaging includes obtaining a dataset volume for an organ of a patient in an ultrasound imaging mode, and providing a template associated with the dataset volume to an optical ultrasound system location. The method also includes providing a virtual transducer with a label thereon to the optical ultrasound system location. A webcam is attached to the template. The webcam is positioned to view the label on the virtual transducer. The method includes determining the planar position of the virtual transducer from optical information obtained from the webcam image of the label. Once the planar position is determined, the planar position of the virtual transducer is related to the corresponding plane in the dataset. The plane related to the dataset is then displayed at the display at the optical ultrasound system location. In some embodiments, the image from the dataset is marked with a plurality of anatomical points within the obtained dataset. In some embodiments, providing the template includes sending a pdf image of the template to the optical ultrasound system location. In still other embodiments, providing the virtual transducer includes sending a pdf image of the virtual transducer to the optical ultrasound system location. Providing the label for the virtual transducer, in some embodiments, includes sending a pdf image of the label to the optical ultrasound system location. This is advantageous in that components of the optical ultrasound system that are not readily available can be sent via E-mail to the location where an optical ultrasound system. The method also includes giving feedback regarding the position of the virtual transducer with respect to the template. Feedback is obtained from the image shown on a display. Further feedback can be provided by an instructor which is live or can be recorded. The method also includes providing a plurality of templates and access to datasets related to the plurality of templates to the optical ultrasound system. Practicing on a plurality of templates allows the student to gain confidence. In addition, it allows the student to act intuitively and encourages exploration. The student also is exposed to the variations that exist amongst patients through the various datasets that are related to a plurality of patients. Access to plurality of datasets can be provided over a connection to the internet. The datasets can be stored in the cloud or at a remote server. The datasets can also be downloaded and stored on a computer at the OPUS site. A template is provided for each of the various datasets. The datasets and the template form a simulation case. The template is labeled with the file name of the dataset so that the appropriate dataset is used in a simulation.

A training device for ultrasound imaging includes a virtual transducer having a first major surface and a second major surface. An optically readable label is positioned on one of the first major surface or the second major surface of the virtual transducer. The optically readable label is adapted to be read by a camera, such as a webcam, in one embodiment. The training device can be read through six degrees of freedom of movement of the virtual transducer. In one embodiment, the other of the first major surface or the second major surface includes labels related to the six degrees of freedom through which the virtual transducer can be moved. The other of the first major surface or the second major surface can also include indicators for positioning the virtual transducer.

Software or a set of instructions for determining position of the virtual transducer in a plane can be stored locally on the computer or can be stored in a server or on the cloud. Similarly, software for translating the planar position of the virtual transducer to a plane within the dataset of a case can be stored on the local machine or stored at a server or on the cloud.

### BRIEF DESCRIPTION OF THE DRAWINGS

FIG. 1 is a schematic drawing of a system that captures and manipulates images, according to an example embodiment.

FIG. 2 shows a schematic view of an ultrasound machine or equipment, according to an example embodiment.

FIG. 3 shows a optical ultrasound simulation site, according to an example embodiment.

FIG. 4 is a perspective view of a virtual transducer that includes a specialized label, according to an example embodiment.

FIG. 5 is a top view of a template associated with a simulator case and associated data set, according to an example embodiment.

FIG. 6 is a view of the optical ultrasound simulation system in use, according to an example embodiment.

FIG. 7 is a flowchart of a training method, according to an example embodiment.

FIG. 8 shows a schematic diagram of a computer system used in the system for driving business, according to an example embodiment.

FIG. 9 is a schematic drawing of a machine readable medium that includes an instruction set, according to an example embodiment.

### DESCRIPTION

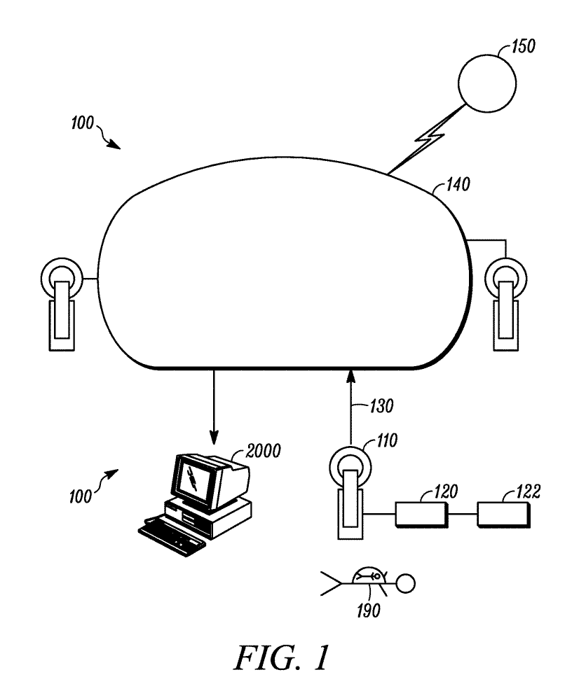

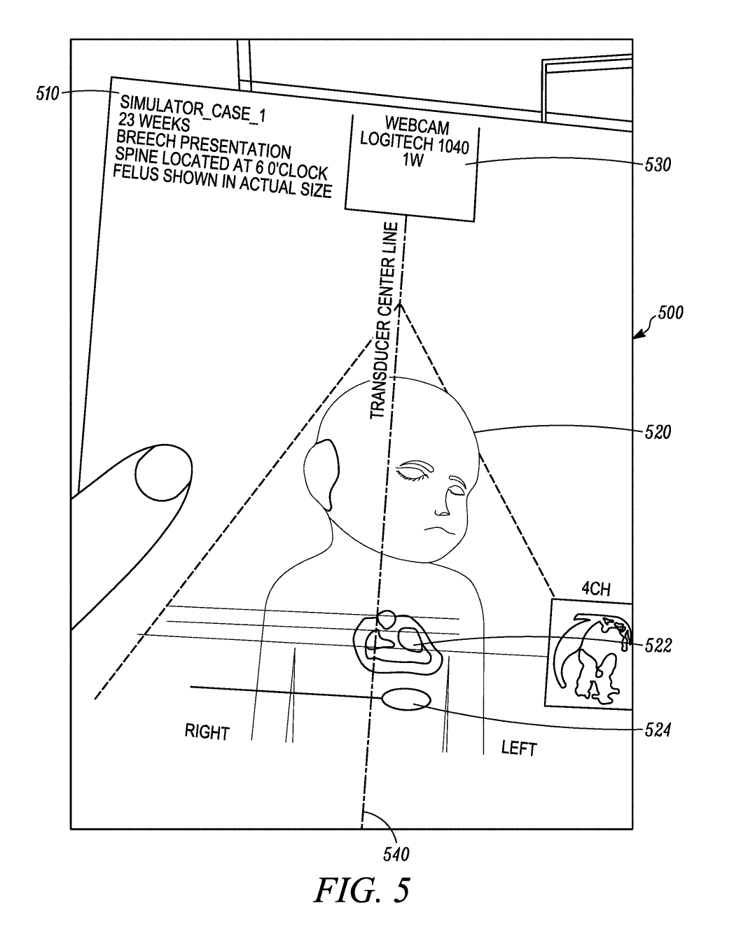

FIG. 1 is a schematic drawing of a system 100 that captures and manipulates images, according to an example embodiment. The system 100 includes an image capture device 110 capable of capturing data related to an image of various body parts of a patient. The system 100 also includes a processor 120 and memory 122 for processing the data obtained from the image capture device 110 and converting the data to useful information such as useful images. In some embodiments, the processor 120 can be a graphical processor or a graphical processing unit, which is adapted to efficiently handle the data obtained and convert it to useful information, such as images. The processor 120 can also convert the image data and information into various formats, such as DICOM or similar information. The system 100 can also include an interface 130 to a network 140, such as an inter-hospital network, and inter-clinic network, a wide area network, a phone network, the internet, or the like. The interface 130 may not provide a direct interface to some networks. For example, the interface 130, in one embodiment, is to an inter-hospital network. The inter-hospital network can have a link to the internet. As a result, the interface 130 can connect to the internet 150 or other network via the inter-hospital network. In another embodiment, the interface may be directly to the internet. It should be noted that the image capture device 110 can be used to perform a number of modalities. In the discussion that follows, the modality discussed is an ultrasound operating with spatio-temporal image-correlation (STIC). In one embodiment, the image capture device 110 is an ultrasound machine, and STIC is one of the modalities in which an ultrasound machine operates. The system 100, as shown in FIG. 5, is taking an image of a fetal heart. Of course, the image capture device can be any image capture device that works with ultrasound technology or any other image capture technology.

Ultrasound technology is an efficient and accurate way to examine and measure internal body structures and detect bodily abnormalities. Ultrasound technology works by emitting high frequency sound waves into a region of interest. The sound waves are emitted from a probe, strike the region of interest, and then reflect back to the probe. For example, certain sound waves strike tissues or fluid in the region of interest before other sound waves do and are thus reflected back to the probe sooner than other sound waves. The ultrasound machine measures the difference in time for various ultrasonic waves to be emitted and reflected back to the transducer probe and produces a picture of the region of interest based on those time differences.

Besides producing an image of the region of interest, ultrasound is capable of determining the velocity of moving tissue and fluids. For example, an ultrasound user can observe a patient's blood as it flows through the heart, determine the speed or flow rate of the bloods movement, and whether the blood is moving towards or away from the heart.

There are three types of Doppler ultrasound:

Color Doppler uses a computer to convert Doppler measurements into an array of colors to show the speed and direction of blood flow through a blood vessel. Doppler ultrasound is based upon the Doppler effect. When the object reflecting the sound waves is moving, it changes the frequency of the echoes that are reflected back to the probe. A Doppler ultrasound machine measures the change in frequency of the sound wave echoes and calculates how fast a particular object is moving within the region of interest. Doppler color flow mapping utilizes color to depict the directional movement of tissue and fluid (such as blood) within the region of interest. Color flow mapping produces a two-dimensional image in color with flow towards the probe shown in one color and flow away from the probe shown in another color.

Power Doppler is a newer technique that is more sensitive than color Doppler and capable of providing greater detail of blood flow, especially when blood flow is little or minimal. Power Doppler, however, does not help the radiologist determine the direction of blood flow, which may be important in some situations. Power Doppler imaging is similar to color flow mapping in that is can produce an image that shows the presence or absence of blood flow and the directional movement of the flow. Power Doppler is advantageous because it is up to five times more sensitive in detecting blood flow and other forms of tissue and fluid movement than color mapping. But, power Doppler imaging is not used to determine the velocity of the moving tissue and fluid.

Spectral Doppler displays blood flow measurements graphically, in terms of the distance traveled per unit of time, rather than as a color picture. It can also convert blood flow information into a distinctive sound that can be heard with every heartbeat.

Ultrasound examinations can help to diagnose a variety of conditions and to assess organ damage following illness. Ultrasound is used to help physicians evaluate symptoms such as: pain, swelling and infection. Ultrasound is a useful way of examining many of the body's internal organs, including but not limited to the:
spleen
pancreas
kidneys
bladder
uterus, ovaries, and unborn child (fetus) in pregnant patients
eyes
thyroid and parathyroid glands
scrotum (testicles)
brain in infants
hips in infants
spine in infants
heart and blood vessels, including the abdominal aorta and its major branches.
Liver
gallbladder

Ultrasound is also used to:
image the breasts and guide biopsy of breast cancer
guide procedures such as needle biopsies, in which needles are used to sample cells from an abnormal area for laboratory testing
diagnose a variety of heart conditions, including valve problems and congestive heart failure, and to assess damage after a heart attack. Ultrasound of the heart is commonly called an “echocardiogram” or “echo” for short.

Doppler ultrasound images can help the physician to see and evaluate:
blockages to blood flow (such as clots)
narrowing of vessels
tumors and congenital vascular malformations
less than normal or absent blood flow to various organs
greater than normal blood flow to different areas which is sometimes seen in infections

With knowledge about the speed and volume of blood flow gained from a Doppler ultrasound image, the physician can often determine whether a patient is a good candidate for a procedure like angioplasty. As can be seen from the above discussion, ultrasound examinations can be widely used for various noninvasive diagnostic tests. Therefore, it would be advantageous to be able to train students to effectively use ultrasound equipment. Furthermore, it would be beneficial to provide training tools without having to physically send them to the students.

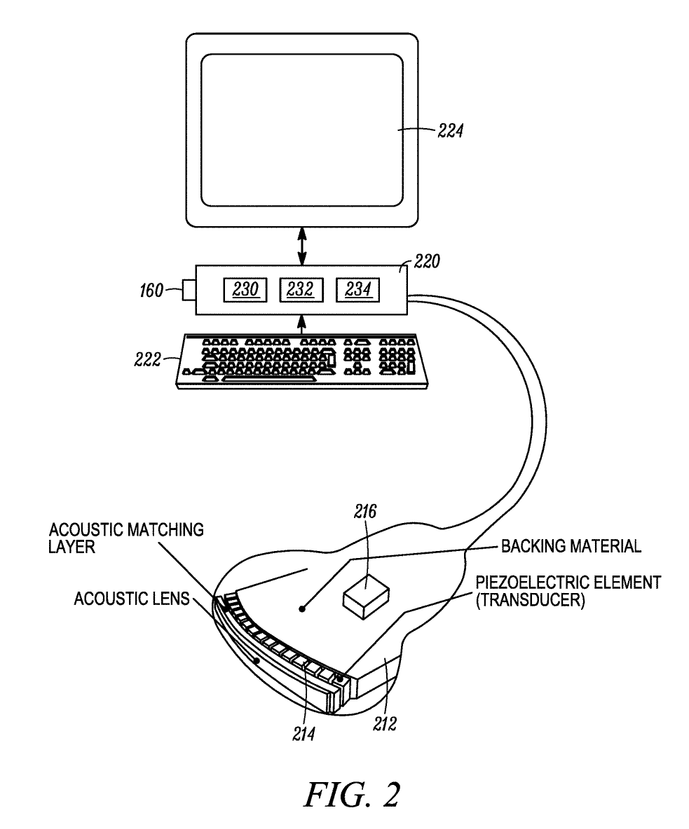

FIG. 2 shows a schematic view of an ultrasound machine or equipment 200, according to an example embodiment. Ultrasound equipment 200 used for ultrasound imaging and treatment can be divided into three main components. First, there is a peripheral ultrasound system 210 that includes a probe 212 with a transducer array 214 or a single element for emitting ultrasound waves. Many times the probe is referred to as a transducer. The peripheral ultrasound system 210 also includes equipment 216 includes signal processing electronics that produces and conditions the ultrasound waves for emission from the probe 212. The ultrasound equipment 200 also includes a host computer system 220 connected to the peripheral ultrasound system 210. The host computer system 220 serves as an interface with the ultrasound user. Specifically, the host computer 220 comprises a keyboard or other input device 222 for controlling the ultrasound equipment 200. The host computer 220 also includes a monitor 224 to display the image to the user.

A microprocessor 230 is within, or connected to or otherwise communicatively coupled to the host computer 220. The microprocessor 230 performs some or all the computing tasks to covert the data collected at the peripheral ultrasound system 210 into the images shown on the monitor 224 to the user. In a Doppler ultrasound system with color flow mapping, the microprocessor 230 will process all the data and generate the velocities of the moving tissues and fluid as well as associated colors to show the directional movement of the tissues and fluid.

To properly process this data, the microprocessor 230 comprises memory 232 and software 234. The software 234 utilizes various algorithms to measure the velocity and to chart the color of the tissue and fluid to depict the directional movement of the tissue and fluid. The software 234 can be stored locally on the host computer 210, can be stored in a peripheral storage device or can be stored in a cloud storage on a server communicatively coupled to host computer. The host computer can download the software from the cloud storage location for storage in the memory of the host computer 220.

As an overview, the imaging system 100 includes a processor 120, 230 that executes an instruction set that causes the computing system to perform operations includes obtaining dataset volumes of various organs. In the specific example of ultrasound equipment 200, the microprocessor 230 executes the software 234 to produce a dataset for images for target organs, such as a heart. In some embodiments, markers are placed within the dataset. From the dataset, selected images of a target organ, such as the heart are produced. Some of the images are produced based on the markers in the dataset which correspond to specific points in the anatomy of the target organ. The imaging system 100, 200 also includes a memory 122, 232 for storing the images, and a display 224 for displaying the images. The memory 122, 232 is capable of holding entire data sets associated with a patient. The data set is all the data collected from a particular patient for a particular study. For example, a pregnant woman generally will have a ultrasound scan conducted to check on the health of the baby. The probe or transducer 212 is moved over the woman's stomach area to check out various organs of the baby. All or some of the data will be held as a dataset. From the dataset, a plurality of planes or planar views can be reconstructed. During most examinations, the sonographer is looking for very specific planes that yield a large amount of diagnostic information. Many times, these planes will be marked. In some instances, the planes are “standard” planes that include 1, 2 or 3 anatomical features. In many embodiments, the memory is capable of holding a multiplicity of data sets from a plurality of patients. The imaging system, and more specifically the image capture device 110, can further include a port 160 communicatively coupled to a network, such as an intra-hospital or intra-clinic network. The port 160 can also be to a telephone network, a wide area network, the internet, or the like. The port 160 can also be to a network that includes web based storage or computing. In other words, rather than storing data or software locally, it can be stored in the cloud. Any computer with access to internet can be enabled to connect through the port to the imaging system 100.

Various embodiments of the invention include producing a number of cases for use in training. These will stem from datasets of patients of the target organs on which the student will be trained. These datasets will be stored on memory which can be accessed from any computer. In the following FIGs. and discussed below is a dataset for a fetus within the womb which is in the breach position. Again, this is just one dataset associated with one case. Other organs can also be the subject of the datasets so that students can learn to conduct ultrasounds to determine if a patient is a good candidate for angioplasty, or for imaging a gallbladder or the like. The various embodiments of the invention allow a student at a remote site to produce a template associated with the training case. Also produced at a remote site is an image which can be attached to a virtual transducer. The virtual transducer can even be produced remotely by either using a 3D printer or by printing out a pattern and producing a virtual transducer at the remote location from locally available material. The template can be placed on a surface. A webcam can also be placed with respect to the template. The relationship in terms of placement between the webcam can be determined. For example, the exact placement between the template and the webcam can be calibrated. In other embodiments, the webcam and the surface can be provided so the distances between these two is known. Once the relation between the webcam and the template is determined, the webcam can visually determine the exact positon of at least three points and therefore a plane. The plane can then be related to the dataset associated with the case. The student can move the virtual transducer over the template until certain planes needed for diagnositics are obtained. The virtual transducer will have six degrees of freedom. The student can move the virtual transducer until an appropriate view is obtained in the educational case. The virtual transducer can be tilted, twisted, moved forward, moved back and moved side to side until a particular image is obtained. In this way, the student is able to get “on the job” training on a virtual ultrasound that can be set up in remote locations. Once the position of the plane of the virtual transducer is determined, the plane is related to the corresponding plane in the dataset associated with the case.

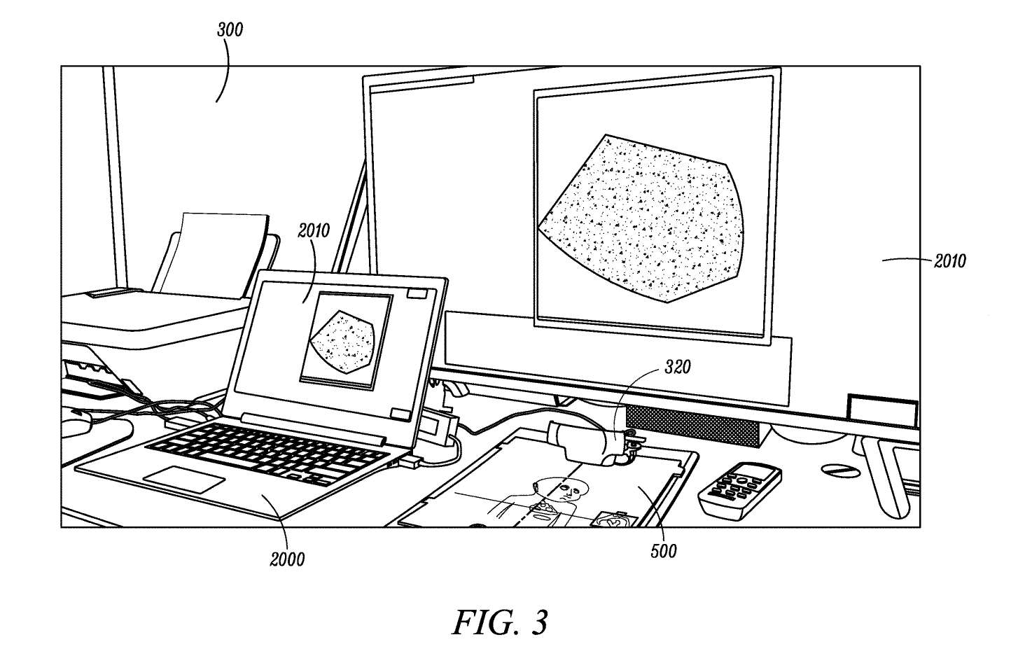

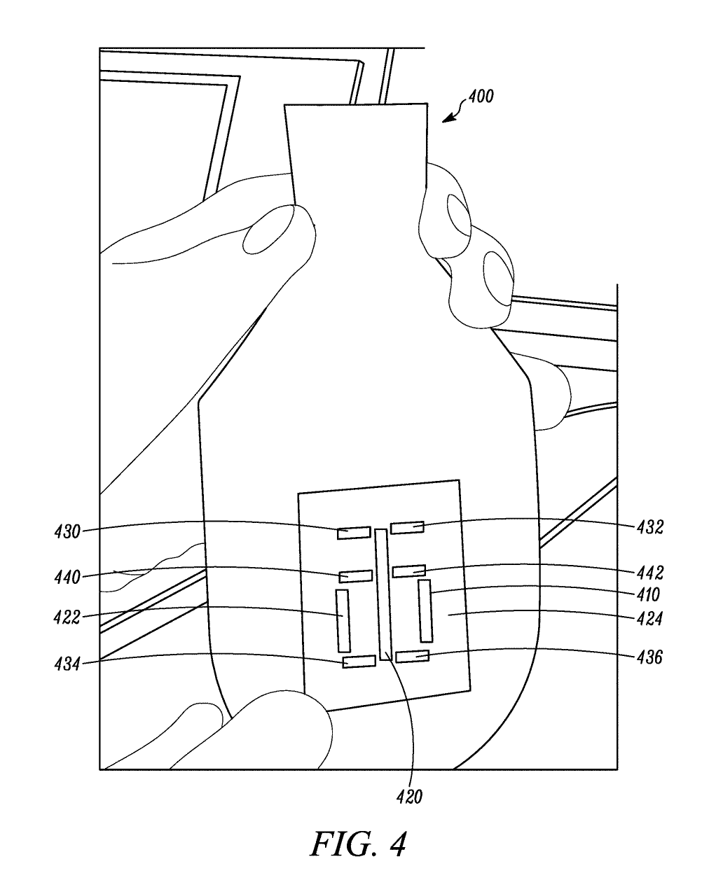

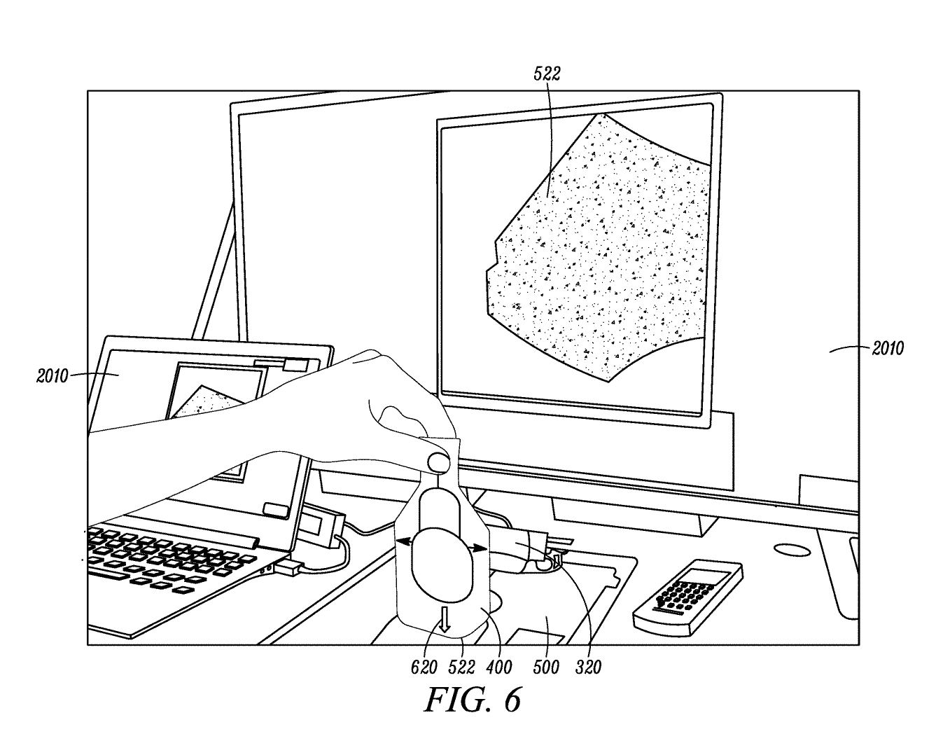

FIG. 3 shows an optical ultrasound simulation site 300, according to an example embodiment. The optical ultrasound simulation 300 can be used for training of sonographers. The optical ultrasound simulation site 300 includes a computer 2000, a webcam 320, a template 500 and a virtual ultrasound transducer 400 (shown in FIGS. 4 and 6). The optical ultrasound simulation site 300 may also be termed as training site or training system for sonographers. In short, a virtual ultrasound transducer 340 and a template 500 are substituted for an actual transducer 212 and a patient 190. The webcam 320 records the position of the virtual ultrasound transducer 340 with respect to the template 500. The plane at which the virtual ultrasound transducer 340 is in can be determined from the image from the webcam 320. The computer 2000 is a general purpose computer that is communicatively coupled to the internet. The webcam 320 is a peripheral device that can be communicatively coupled to the computer 2000 through a port, such as a port for connecting to a uniform serial bus. The webcam can be one that is readily available, such as a Logitech C615 HD Webcam offers HD 720p video calling and Full HD 1080p video recording and sharing. It is a versatile and portable and is a 1080p Full HD webcam. Logitech is a company headquartered in Lausanne, Switzerland with offices at 165 University Avenue, Palo Alto, Calif. The image on the webcam of the virtual ultrasound transducer 340 can be measured for distance as well as planar orientation. The planar orientation can be then converted to a planar orientation a dataset associated with the simulation case. The dataset will generally be housed in a server remote from the optical ultrasound simulation site 300. The computer 2000 has a display 2010. The display shows the corresponding plane in the dataset. As shown in FIG. 3, there is a small display 2010 associated with the computer 2000 (a laptop is used in this embodiment) and another larger display 2010 that is communicatively coupled to the computer 2000. Both displays carry or show the same image in this instance.

As shown in FIG. 3, the template 500 is placed on a surface along with the webcam 320. The position of the webcam 320 with respect to the template 500 is determined. In the embodiment shown, a clipboard has been used. In this particular embodiment, the webcam is attached to the clip of the clipboard and the template is carefully placed on the clipboard. In this instance, the webcam and the clipboard have a known relationship with respect to distances. The template 500 is provided with certain lines that relate to planes of a baby's body. One of these lines can be designated for the purpose of calibrating the distance relation between the template 500 and the webcam 320. The webcam 320 is connected to the computer 2000. The computer 2000 is provided with simulation software. This simulation software can be delivered to the user, in some embodiments, using a dongle or portable memory. In other instances, the computer 2000 can be loaded with software from a server somewhere else in the world via an internet connection. In still a further embodiment, the software can be loaded into the cloud and the computer 2000 can use the software instruction set from the cloud as the simulation software.

In one embodiment, portions of the optical ultrasound simulation site 300 are sent to the remote site. If delivery is unreliable, persons at the remote site can still be equipped as an optical ultrasound simulation site 300. All that is needed is a computer, a webcam, and a printer. The template for a particular simulation case can be sent to the optical ultrasound simulation site 300. A pattern for a virtual transducer can also be sent as a PDF to the optical ultrasound simulation site 300. From the pattern, local materials can be used to form a virtual transducer. A sticker can also be provided on the pattern for the virtual transducer. This “sticker” is used to measure distances and the orientation of the virtual transducer. Thus, an optical ultrasound simulation site 300 can be set up in very remote areas. There is also no need to go through customs or the like. An optical ultrasound simulation site 300 can be set up in minutes or hours provided a computer, webcam and an internet connection are available.

FIG. 4 is a perspective view of a virtual transducer 400 that includes a specialized label or sticker 410, according to an example embodiment. The transducer 400 shown is provided to the user at the optical ultrasound simulation site 300. In this particular embodiment, the virtual transducer 400 is made from PVC plastic and shipped to the optical ultrasound simulation site 300. The virtual transducer 400 could be made of any material, such as plastic, cardboard, wood or the like. Basically, the virtual transducer could be fashioned from any material including local materials in places where delivery of components might not be reliable. It is contemplated that a pdf file that includes the outline of a virtual transducer 400 could be sent to a remote location. The specialized label 410 could be part of the pdf. A person on the receiving end could trace the outline of the virtual transducer 400 and carve wood or pour plastic to form the virtual transducer 400. In another embodiment, the virtual transducer can be printed on a 3D printer. A label 410 can be applied to the printed part. A definite advantage of the system 300 is that many of the specialized parts that convert a computing device into an optical ultrasound system can be sent as a pdf and printed at a remote site. The label 410 and an outline of the virtual transducer 400 can be sent as pdf files to a remote site. In some instances a 3D printable file can be sent to the remote site so that a virtual transducer 400 can be printed at the remote site as a useable part.

The label 410 will now be further detailed. The label 410 includes a dark background 412 with a number of contrasting lines in a specific pattern. The label 410 includes a central axis or vertical line 420. Another vertical line 422 is substantially parallel to the vertical axis and positioned left of the central or vertical axis 420. Yet another vertical line 424 is substantially parallel to the vertical axis and positioned right of the central or vertical axis 420. The label 410 includes three pairs of horizontal lines which are substantially perpendicular to the central or vertical axis 420. Lines 430 and 432 are positioned near the top of the pattern. Lines 434 and 436 are positioned near the bottom of the pattern. Lines 440 and 442 are positioned between lines 430 and 432 and lines 434 and 436. The lines 440 and 442 are placed away from the exact midpoint between the top and bottom of the pattern.

By viewing the pattern using the webcam 320, the distance between the webcam and the label can be determined. In addition, the distance between the lines, which is known, can be measured to determine the angles associated with the plane in which the virtual transducer is being held. For example, if the virtual transducer 400 is twisted about the y axis or cable axis, the distance between 424 and 422 will be closer together. If the virtual transducer is moved along the roll axis, the distance between lines 434 and 422 will be closer. In short, by measuring the distances between various sets of lines, the angles at which the plane is held can be determined. The angle of the planar surface of the label 410 is also the angle of the virtual transducer 400. In some instances, there may be a set offset between the label 410 and a parallel plane centered between the two major surfaces of the virtual transducer 400.

In another embodiment, a set of circles could form a pattern. The circles could be used to determine three points on the plane of the label. A parallel plane between the major surfaces of the virtual transducer could then be determined and translated into the plane of the organ or baby being scanned.

FIG. 5 is a top view of a template 500 associated with a simulator case 510 and associated data set (shown as memory 122, 232 in FIGS. 1 and 2, respectively), according to an example embodiment. The template 500 includes a reference to the case number, which is shown in the upper left-hand corner of the template 500. The case number is set forth as a file name 510. The file name 510 is the same as the file name for the data set in memory 122, 232 or stored on a remote server or in the cloud. Also included in the upper left-hand corner is a brief description of the case. As shown, this case includes a fetus at 23 weeks which is in a breech position. An outline of the fetus 520 is shown. Also shown within the outline of the fetus 520 are some of the major organs. In this particular embodiment, the heart 522 is shown as well as the stomach 524. These are some of the major organs that are typically scanned during prenatal checkups. The template 500 also includes a space 530 for the WebCam 320. In the particular embodiment shown the brand name of the WebCam as well as the capabilities or needed capabilities of the WebCam are also set forth at this space 530. The space 530 is used to position the WebCam 320 on the template 500. The template 500 also includes several lines which correspond to diagnostic planes for the fetus 520. For example, one line is labeled “4cH” and passes through the heart 522 of the fetus 520. This is a line corresponding to the plane which passes through the four chambers of the heart 522. The virtual transducer or probe 400 can be placed on this line. The WebCam 320 will note the distance and show the corresponding plane, that is to say the plane with four chambers of the heart, on the display 2010. Other lines in the template 500 show or depict the position of planes which correspond to other diagnostic planes of interest. Also included on the template 500 is a transducer centerline 540. This enables the operator or trainee to place the transducer 400 in the appropriate center position on a particular line to find a plane of interest for diagnostic purposes.

FIG. 6 is a view of the optical ultrasound simulation system 300 in use, according to an example embodiment. In this particular embodiment, a user or student is holding the probe or transducer 400 over a specific line on the template 500. It appears that the transducer 400 is being held on a line that shows two chambers of the heart 522 on the monitor 2010. The label 410 is positioned on the major surface of the transducer 400 that faces the WebCam 320. In other words, in this view we are seeing the other major surface of the virtual transducer 400. The center portion of the virtual transducer 400 is depicted by arrow 620. This arrow 620 corresponds to the vertical centerline 420 of the label 410. The major surface shown also includes arrows labeled left and right for side to side movements as well as of and down. These are two of the degrees of freedom through which the virtual transducer 400 can be moved. In total, there are 6 degrees of freedom through which the virtual transducer can be moved. Amongst the 6 degrees of freedom is rotation about the cable axis, rotation about a tilt axis or roll axis, rotation about a yaw axis, and rotation of the virtual transducer 400. During these motions or movements the optical ultrasound technology works using a sign pattern 410 printed on a paper “probe” or virtual transducer 400. The webcam 320 reads the sign pattern for 10 to track the “probe” or virtual transducer 400 position in space. Based on the position of the virtual transducer or “probe” the optical ultrasound system (OPUS) extracts a specific plane from a preloaded 3D or 4D volume or data set from memory 122, 232. OPUS then shows the specific plane as a 2D image to mimick or simulate a 2D ultrasound scanning. Each template is labeled as “OPUS_CASE_1 . . . 2 . . . ” and corresponds to a 3D volume dataset with the same naming stored in memory 122, 232 or at a remote server or in the cloud. There are a number of interchangeable or exchangeable datasets that can be used for training purposes. Each of the exchangeable set of templates show a particular position of the fetus, or the organ to be scanned

In some instances, a webcam 320 with a given optical aperture, may only detect a limited range of angles of the virtual transducer 400. Transverse-Longitudinal views may be not available directly. The user can overcome this limitation by loading a tilted version of the same case, or by pushing a tilting button that rotates the model

OPUS 300 is designed to teach or practice a focalized anatomy or organ. This is a “regional” scanning method, and not “panoramic” as with other ultrasound simulators

Certain lightning conditions may produce jumps or loss of detection of the printed sign or label 410 which may lead to erratic performance Diffuse lighting with no spots, is preferred to avoid any shadowing over the pattern or specialized label 410

Three modes of operation are contemplated:

OPUS SOLO: For “freestyle” navigation of the anatomy, emulating the scan of a patient, with the goal of achieving a target reference standard plane. OPUS SOLO lets or allows the student user to become familiar with scanning by unsupervised use. In this mode of operation, two windows are shown. One of the windows is for the 2D live scanning, and another smaller window shows the target reference image.

OPUS MAESTRO: Brings the possibility of being supervised by an expert while performing predefined exercises directed towards obtaining standard planes or the planes most commonly used for medical diagnostic purposes. The student user receives understandable tips and recommendations in a visual and natural voice manner In the OPUS MAESTRO mode there are three more windows available for the user:
MONITORING GUIDES: These 3 views from different sides will make easy to understand exactly how the probe or virtual transducer 400 is located with respect of the target image. A set of monitoring guides illustrate independently the misalignments of each one of the possible six hand movements. In this way, the student user can quickly spot the problems with their technique and correct those problems.

CUBE VIEW: ANATOMICAL VOLUME displayed in perspective as seen by the user student. With this view, the student user can realize how the 2D plane is located within the “patient” dataset block, and where is the target destination plane inside the same cube. Users will visualize the possible actions to take in order to reach the required destination, by the visualization of four connecting lines (red color). As the user gets closer to the target plane the red connecting lines will be shorter until they disappear completely when touching four corner blue balls.

VIDEO TUTORIALS: These contextual aids will show what movement to make next. This view shows a probe or virtual transducer 400 and the specific movement the user has to make expressed as TWIST/FAN/HOUR rotations, and UP-DOWN/Left-Right/Push-Pull parallel shift displacements. These are the six degrees of freedom that the user student has.

AUDIO TUTORIALS: The same information is provided with clear, not annoying, voice messages. When the target is obtained an OK sign is shown.

OPUS SUITE: It allows a professor to generate a set of TARGET PLANES and embed them into each case file. OPUS will generate a log to qualify how a student performs the navigation lessons. Qualities such as quantity of movements, hesitance, accuracy of final planes, total time for each target achieved are recorded so that progress can be monitored on a per student user basis.

In some embodiments, OPUS can be programmed to be more relaxed on certain movements and target precision ranges. This allows for instance the tolerance of HOUR position, or side locations of a given plane, as if in real patient situations. The teacher or professor is also allowed to make stricter targeting requests

OPUS has some very distinct advantages. For many years ultrasound education has been a challenging task to accomplish. Different students obtain different skills at different rates. Mastering the skills needed may take years of practice and require years of scanning on the job. There have been problems with learning on the job. There is difficulty in putting theory into practice. Connecting concepts with real life needs is difficult. Intuitive learning is limited. Intuitive learning is not developed through a wide range of potential and clinical possibilities. The “comfort zone” is not expanded because “pushing for more” is not necessarily rewarded. OPUS cures these shortcomings since the student is free to try new things, to push the envelope and there are no consequences or failures. The student develops good hand-eye coordination based on practice with a variety of cases. The student learns the effects of various hand movements

OPUS provides a plurality of exercises and successful completion of these exercises allows the student to gain the necessary skills and stretch the comfort zone of learning without a downside. An expert supervisor can qualify the obtained results and provide for accurate feedback. An expert supervisor can also alert the student user with respect to errors during the learning/practicing process

Using OPUS many of the problems associated with learning ultrasound imaging can be overcome. Standard images can be knowingly obtained. Patient variations can be introduced so the student sees these variations and ultimately can obtain the necessary standard images with confidence despite patient variations, fetal growth, and other artifacts. Using OPUS instills 3D thinking The student learns about the six degrees of freedom, anatomy navigation plans, and all about a movement set for a given fetal data set. The student learns to look for abnormalities and gain confidence in their results.

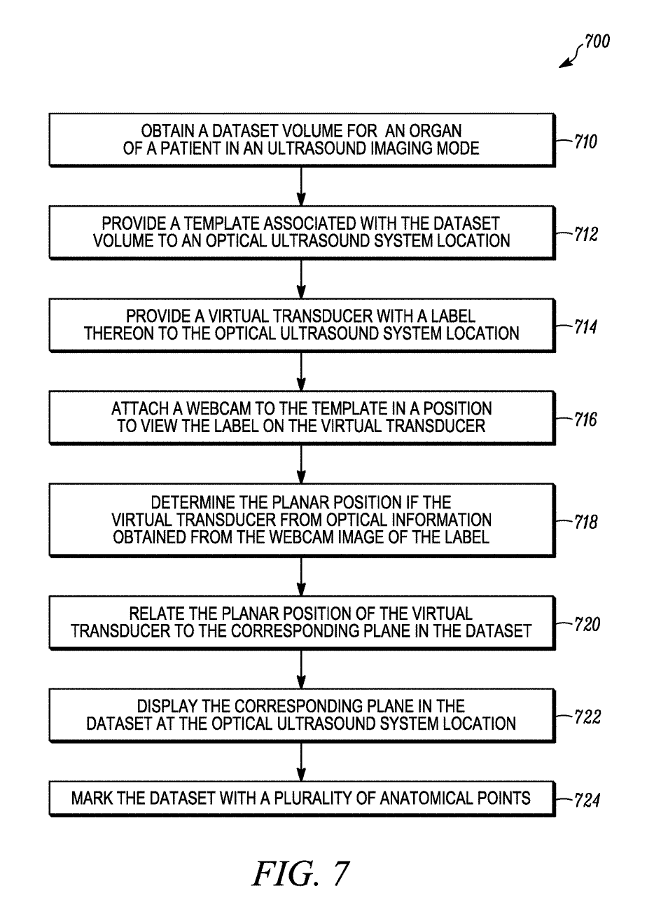

FIG. 7 is a flowchart of a training method 700, according to an example embodiment. The training method 700 for imaging includes obtaining a dataset volume for an organ of a patient in an ultrasound imaging mode 710, and providing a template associated with the dataset volume to an optical ultrasound system location 712. The method 700 also includes providing a virtual transducer with a label thereon to the optical ultrasound system location 714. A webcam is attached to the template. The webcam is positioned to view the label on the virtual transducer 716. The method 700 includes determining the planar position of the virtual transducer from optical information obtained from the webcam image of the label 716. Once the planar position is determined, the planar position of the virtual transducer is related to the corresponding plane in the dataset 720. The plane related to the dataset is then displayed at the display at the optical ultrasound system location 722. In some embodiments, the image from the dataset is marked with a plurality of anatomical points within the obtained dataset 724. In some embodiments, providing the template 712 includes sending a pdf image of the template to the optical ultrasound system location. In still other embodiments, providing the virtual transducer 714 includes sending a pdf image of the virtual transducer to the optical ultrasound system location. Providing the label for the virtual transducer, in some embodiments, includes sending a pdf image of the label to the optical ultrasound system location. This is advantageous in that components of the optical ultrasound system that are not readily available can be sent via E-mail to the location where an optical ultrasound system. The method also includes giving feedback regarding the position of the virtual transducer with respect to the template. Feedback is obtained from the image shown on a display. Further feedback can be provided by an instructor which is live or can be recorded. The method also includes providing a plurality of templates and access to datasets related to the plurality of templates to the optical ultrasound system. Practicing on a plurality of templates allows the student to gain confidence. In addition, it allows the student to act intuitively and encourages exploration. The student also is exposed to the variations that exist amongst patients through the various datasets that are related to a plurality of patients. Access to plurality of datasets can be provided over a connection to the internet. The datasets can be stored in the cloud or at a remote server. The datasets can also be downloaded and stored on a computer at the OPUS site. A template is provided for each of the various datasets. The datasets and the template form a simulation case. The template is labeled with the file name of the dataset so that the appropriate dataset is used in a simulation.

A training device for ultrasound imaging includes a virtual transducer having a first major surface and a second major surface. An optically readable label is positioned on one of the first major surface or the second major surface of the virtual transducer. The optically readable label is adapted to be read by a camera, such as a webcam, in one embodiment. The training device can be read through six degrees of freedom of movement of the virtual transducer. In one embodiment, the other of the first major surface or the second major surface includes labels related to the six degrees of freedom through which the virtual transducer can be moved. The other of the first major surface or the second major surface can also include indicators for positioning the virtual transducer.

Software or a set of instructions for determining position of the virtual transducer in a plane can be stored locally on the computer or can be stored in a server or on the cloud. Similarly, software for translating the planar position of the virtual transducer to a plane within the dataset of a case can be stored on the local machine or stored at a server or on the cloud.

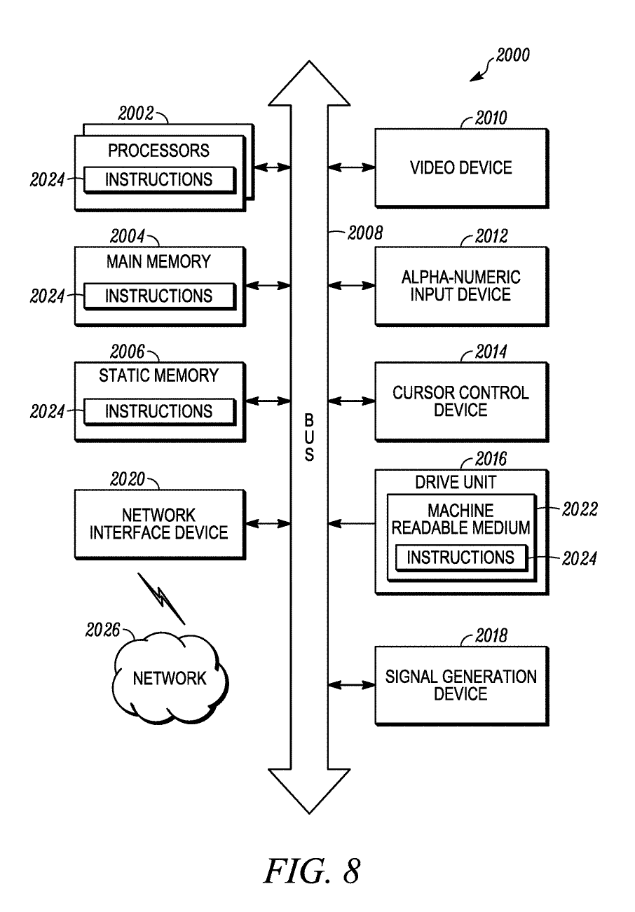

FIG. 8 shows a diagrammatic representation of a computing device for a machine in the example electronic form of a computer system 2000, within which a set of instructions for causing the machine to perform any one or more of the error correction methodologies discussed herein can be executed or is adapted to include the apparatus for error correction as described herein. In various example embodiments, the machine operates as a standalone device or can be connected (e.g., networked) to other machines. In a networked deployment, the machine can operate in the capacity of a server or a client machine in a server-client network environment, or as a peer machine in a peer-to-peer (or distributed) network environment. The machine can be a personal computer (PC), a tablet PC, a set-top box (STB), a Personal Digital Assistant (PDA), a cellular telephone, a portable music player (e.g., a portable hard drive audio device such as a Moving Picture Experts Group Audio Layer 3 (MP3) player, a web appliance, a network router, a switch, a bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 2000 includes a processor or multiple processors 2002 (e.g., a central processing unit (CPU), a graphics processing unit (GPU), arithmetic logic unit or all), and a main memory 2004 and a static memory 2006, which communicate with each other via a bus 2008. The computer system 2000 can further include a video display unit 2010 (e.g., a liquid crystal display (LCD) or a cathode ray tube (CRT)). The computer system 2000 also includes an alphanumeric input device 2012 (e.g., a keyboard), a cursor control device 2014 (e.g., a mouse), a disk drive unit 2016, a signal generation device 2018 (e.g., a speaker) and a network interface device 2020.

The disk drive unit 2016 includes a computer-readable medium 2022 on which is stored one or more sets of instructions and data structures (e.g., instructions 2024) embodying or utilized by any one or more of the methodologies or functions described herein. The instructions 2024 can also reside, completely or at least partially, within the main memory 2004 and/or within the processors 2002 during execution thereof by the computer system 2000. The main memory 2004 and the processors 2002 also constitute machine-readable media.

The instructions 2024 can further be transmitted or received over a network 2026 via the network interface device 2020 utilizing any one of a number of well-known transfer protocols (e.g., Hyper Text Transfer Protocol (HTTP), CAN, Serial, or Modbus).

While the computer-readable medium 2022 is shown in an example embodiment to be a single medium, the term “computer-readable medium” should be taken to include a single medium or multiple media (e.g., a centralized or distributed database, and/or associated caches and servers) that store the one or more sets of instructions and provide the instructions in a computer readable form. The term “computer-readable medium” shall also be taken to include any medium that is capable of storing, encoding, or carrying a set of instructions for execution by the machine and that causes the machine to perform any one or more of the methodologies of the present application, or that is capable of storing, encoding, or carrying data structures utilized by or associated with such a set of instructions. The term “computer-readable medium” shall accordingly be taken to include, but not be limited to, solid-state memories, optical and magnetic media, tangible forms and signals that can be read or sensed by a computer. Such media can also include, without limitation, hard disks, floppy disks, flash memory cards, digital video disks, random access memory (RAMs), read only memory (ROMs), and the like.

When the computerized method 1200, discussed above, is programmed into a memory of a general purpose computer, the computer and instructions form a special purpose machine. The instructions, when programmed into a memory of a general purpose computer, are in the form of a non transitory set of instructions.

The example embodiments described herein can be implemented in an operating environment comprising computer-executable instructions (e.g., software) installed on a computer, in hardware, or in a combination of software and hardware. Modules as used herein can be hardware or hardware including circuitry to execute instructions. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software programs for implementing the present method(s) can be written in any number of suitable programming languages such as, for example, Hyper text Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Perl, UNIX Shell, Visual Basic or Visual Basic Script, Virtual Reality Markup Language (VRML), ColdFusion™ or other compilers, assemblers, interpreters or other computer languages or platforms.

The present disclosure refers to instructions that are received at a memory system. Instructions can include an operational command, e.g., read, write, erase, refresh and the like, an address at which an operational command should be performed; and the data, if any, associated with a command The instructions can also include error correction data.

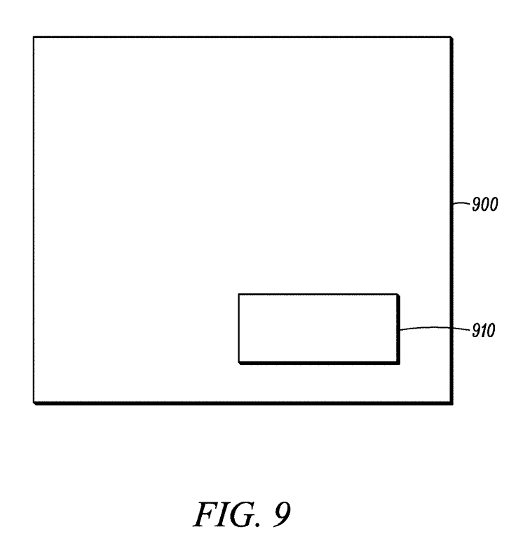

FIG. 9 is a schematic drawing of a non-transitory media, according to an example embodiment. Instruction sets or software can be provided on a non-transitory media 900 that has the instruction set 910 stored thereein.

This has been a detailed description of some exemplary embodiments of the invention(s) contained within the disclosed subject matter. Such invention(s) may be referred to, individually and/or collectively, herein by the term “invention” merely for convenience and without intending to limit the scope of this application to any single invention or inventive concept if more than one is in fact disclosed. The detailed description refers to the accompanying drawings that form a part hereof and which shows by way of illustration, but not of limitation, some specific embodiments of the invention, including a preferred embodiment. These embodiments are described in sufficient detail to enable those of ordinary skill in the art to understand and implement the inventive subject matter. Other embodiments may be utilized and changes may be made without departing from the scope of the inventive subject matter. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A training method for imaging comprising:
obtaining a dataset volume for an organ of a patient in an ultrasound imaging mode;
providing a template associated with the dataset volume to an optical ultrasound system location;
providing a virtual transducer with a label thereon to the optical ultrasound system location;
attaching a webcam to the template, the webcam positioned to view the label on the virtual transducer;
determining the planar position of the virtual transducer from optical information obtained from the webcam viewing of the label; and
relating the planar position of the virtual transducer to the corresponding plane in the dataset.

2. The training method for imaging of claim 1 further comprising displaying the plane from the dataset at a monitor at the optical ultrasound system location.

3. The training method for imaging of claim 1 further comprising marking a plurality of anatomical points within the obtained dataset.

4. The training method for imaging of claim 1 wherein providing the template includes sending a pdf image of the template to the optical ultrasound system location.

5. The training method for imaging of claim 1 wherein providing the virtual transducer includes sending a pdf image of the virtual transducer to the optical ultrasound system location.

6. The training method for imaging of claim 1 wherein providing the label for the virtual transducer includes sending a pdf image of the label to the optical ultrasound system location.

7. The training method for imaging of claim 1 further comprising giving feedback regarding the position of the virtual transducer with respect to the template.

8. The training method for imaging of claim 1 further comprising providing a plurality of templates and access to datasets related to the plurality of templates to the optical ultrasound system.

9. The training method for imaging of claim 8 wherein access to plurality of datasets is provided over a connection to the internet.

10. A training device for ultrasound imaging comprising:
a virtual transducer having a first major surface and a second major surface; and
an optically readable label positioned on one of the first major surface or the second major surface.

11. The training device of claim 10 wherein the label can be read through six degrees of freedom of movement of the virtual transducer.

12. The training device of claim 10 wherein the other of the first major surface or the second major surface includes labels related to the six degrees of freedom through which the virtual transducer can be moved.

13. The training device of claim 10 wherein the other of the first major surface or the second major surface includes indicators for positioning the virtual transducer.

14. Software for determining position of the virtual transducer plane.

15. Software for translating the planar position of the virtual transducer to a plane within the dataset of a case.
